(19) United States
(12) Patent Application Publication
(10) Pub. No.: US 2012/0259121 A1
(43) Pub. Date: Oct. 11, 2012

(54) PROCESS FOR THE PREPARATION OF MONTELUKAST AND SALTS THEREOF

(75) Inventors: Theoharis V. Koftis, Thessaloniki (GR); Ravikant R. Soni, Thessaloniki (GR)

(73) Assignee: PHARMATHEN S.A., PALLINI-ATTIKIS (GR)

(21) Appl. No.: 13/515,893

(22) PCT Filed: Dec. 23, 2009
(86) PCT No.: PCT/EP2009/009249

§ 371 Date: Jun. 14, 2012

(30) Foreign Application Priority Data
Dec. 23, 2009 (EP) PCT/EP2009/009249

(51) Int. Cl. C07D 215/18

(52) U.S. Cl. 546/174

(57) ABSTRACT

The present invention relates to an improved process for the preparation of Montelukast and pharmaceutical acceptable salts or derivatives thereof, in particular to a process for large scale production of Montelukast and salts thereof in high yield and high purity and pharmaceutical preparations containing said compounds.

### TECHNICAL FIELD OF THE INVENTION

The present invention relates to an improved process for the preparation of Montelukast and pharmaceutical acceptable salts or derivatives thereof and in particular to a process for large scale production of Montelukast or salts thereof and pharmaceutical preparations containing said compounds.

### BACKGROUND OF THE INVENTION

Montelukast is chemically designated as (R, E)-2-(1-((1-(3-(2-(7-chloroquinolin-2-yl)vinyl)phenyl)-3-(2-(2-hydroxypropan-2-yl)phenyl)propylthio)-methyl) cyclopropyl) acetic acid and is used in the form of Sodium salt. Montelukast sodium is a known leukotriene receptor antagonist employed for the treatment of asthma. It is also indicated for the relief of symptoms of nasal allergies. Montelukast sodium is presented by the chemical structure of Formula I.

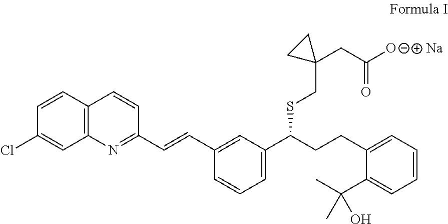

Various methods are already known for the preparation of Montelukast or salt thereof due to its useful properties. Prior art processes for the preparation of Montelukast sodium present the disadvantages of non-satisfactory yield and purity of the product and non-feasibility for large scale production. Furthermore, the compound often comprises significant amounts of unwanted by-products and the reaction may require a long period of time to be completed.

Montelukast and its sodium salt and methods for the preparation thereof, were first disclosed in EP-B-0480717, wherein Montelukast sodium is obtained by hydrolysis of methyl ester of O-protected Montelukast, followed by conversion of the obtained Montelukast free acid into Montelukast sodium. However, this process is not suitable for large-scale production because it requires tedious column chromatographic purification. Moreover, this process needs multiple protection/de-protection cycles and the yield of the product is very low.

EP-B-0737186 discloses a process for the preparation Montelukast sodium wherein no column chromatographic purification is used, said process comprises reaction of crude Montelukast free acid with dicyclohexylamine to form the corresponding salt, purification of said salt to obtain purified Montelukast free acid and conversion thereof into Montelukast sodium. However, the purification of the dicyclohexylamine salt of Montelukast is a long lasting and difficult process, as extra purification steps are required in order to remove the process related impurities, such as dehygration and cyclic ether impurities. In addition, said extra purification steps result in increased production costs and reduced yield of the product.

US-A-2008/0188664 discloses a process for the preparation of Montelukast sodium, wherein the reaction conditions are fine-tined to achieve minimal level of the process related impurities and reasonable yield. Said document also discloses a process of purifying the amine additional salt of Montelukast without forming additional impurities. According to this process, the amount of the process-related impurities in the final product is minimized. However, the yield of the product and the chemical and optical purity of the product are still low.

Although each of the above patents represents an attempt to improve the efficiency of purifying and isolating Montelukast free acid and to prepare highly pure Montelukast sodium, there still exists a need for a cost-effective process for large scale production of Montelukast and the pharmaceutically acceptable salts thereof, which provides higher yield with higher chemical and optical purity.

### SUMMARY OF THE INVENTION

It is, therefore, an object of the present invention to provide an improved process for the preparation of Montelukast or pharmaceutical acceptable salts thereof, which overcomes the deficiencies of the prior art processes and results to a cost effective industrial production without sacrificing the yield and quality of the product.

Another object of the present invention is to provide an improved method for the preparation of Montelukast or salts thereof by using mild and well-controlled reaction conditions, so that the steps for the protection/de-protection of the functional groups can be avoided without sacrificing the yield and quality of the product.

Further object of the present invention is to provide an improved method for the preparation of Montelukast or salts thereof by selecting the appropriate reactants, catalysts, solvent systems and conditions used during the organic reactions, so that the purity (both chemical purity and optical purity) and yield of the reaction are increased and the presence of any contaminants and formed by-products is minimized.

In accordance with the above objects of the present invention, a process for the preparation of Montelukast sodium of Formula I comprising the following steps is provided:

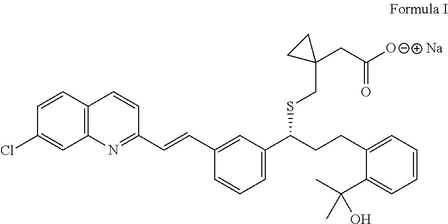

(a) dissolution of {S}-1-[3-[2-(7-chloro-2-quinolinyl)ethyl]phenyl]-3-[2-(1-hydroxy-1-methylethyl)phenyl]propanol of Formula II in a solvent in the presence of catalyst and with addition of mesyl chloride thereto to obtain a reaction mass;

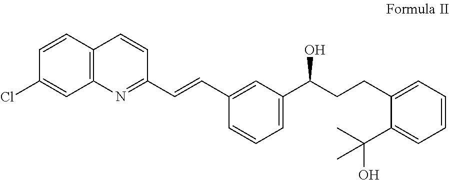

(b) addition of a solution of 2-[1-(mercaptomethyl)cyclopropyl]acetic acid of Formula III into the reaction mass obtained from step (a) followed by a solution of sodium methoxide and work-up to obtain crude Montelukast free acid of Formula IV;

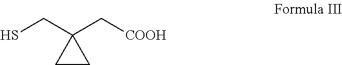

(c) reaction of crude Montelukast free acid of Formula IV with L-ephedrine to obtain Montelukast L-ephedrine salt of Formula V;

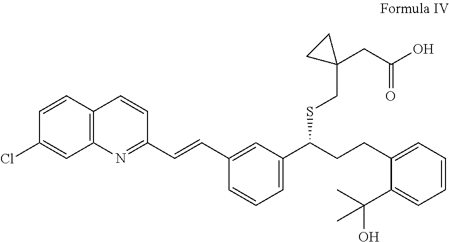

(d) purification of crude Montelukast L-ephedrine salt of Formula V in order to obtain substantial pure Montelukast L-ephedrine salt of Formula V with chemical purity of not less than 99.5% and optical purity of not less than 99.9%;

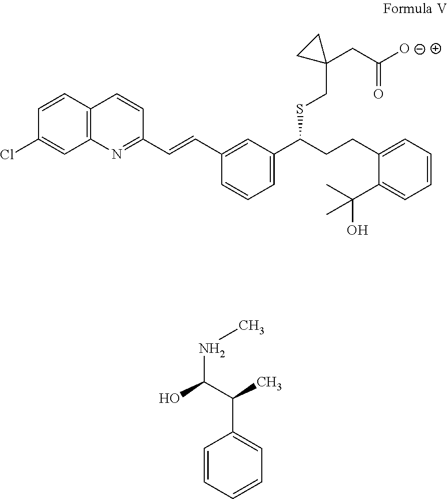

(e) conversion of the substantial pure Montelukast L-ephedrine salt of Formula V into substantial pure Montelukast sodium of Formula I.

Preferred embodiments of the present invention are set out in dependent claims 2 to 14.

Other objects and advantages of the present invention will become apparent to those skilled in the art in view of the following detailed description.

### DETAILED DESCRIPTION OF THE INVENTION

The present invention relates to an improved process for the preparation of Montelukast and pharmaceutically acceptable salts, which is characterized in substantially milder and well-controlled reaction conditions, without sacrificing the yield and quality of the product and low cost of reactants and reagents.

The process of the present invention provides a simplified process for the preparation of Montelukast sodium, wherein the number of the reaction steps is minimized and protection/deprotection steps as used in many prior art processes are avoided. This is attributed to the fine adjustment of the reaction conditions.

In addition, the fine adjustment of the reaction condition of the present invention has also been proved to be useful in minimizing the content of any contaminants and process-related impurities.

The process of the present invention produces highly pure Montelukast sodium with an improved overall yield. This is attributed to the use of an optically pure base, such as L-ephedrine, in the purification process. Montelukast-L-ephedrine salt can be obtained in very good chemical and optical purity. Thus, the Montelukast acid released from the Montelukast-L-ephedrine salt is obtained in very good chemical and optical purity by using simpler purification procedure.

According to the present invention, the process for the preparation of highly pure Montelukast and pharmaceutical salts thereof comprises the following steps:

Stage I: Preparation of crude Montelukast free acid (as shown in Scheme 1).

The preparation of crude Montelukast free acid (Scheme 1) is achieved in a one-pot reaction of compound of formula II with mesyl chloride followed by (1-mercaptomethyl-cyclopropyl)acetic acid of Formula III.

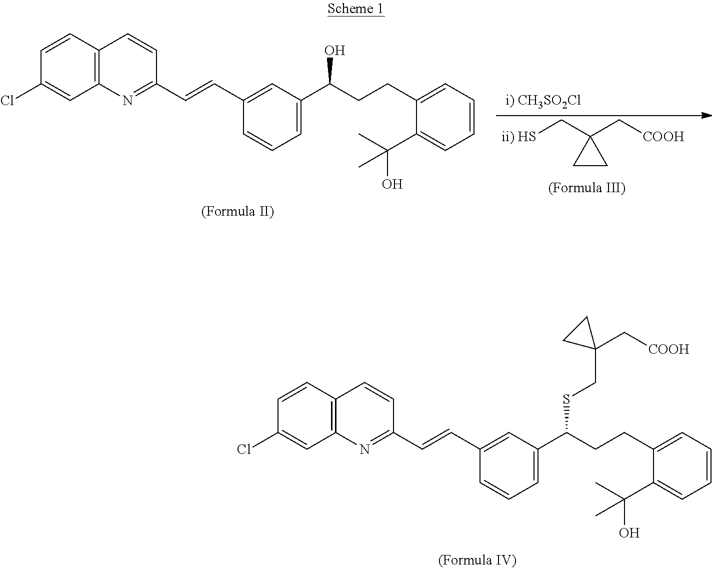

In stage I, a diol compound {S}-1-[3-[2-(7-chloro-2-quinolinyl)ethyl]phenyl]-3-[2-(1-hydroxy-1-methylethyl)phenyl]propanol of Formula II is chosen as the key starting material. Without protecting the two hydroxyl functional groups, this starting material is reacted with mesyl chloride at low temperature in the presence of an amine, preferably a tertiary amine, as catalyst. Upon completion of the mesylation, without isolation of the intermediate, the reaction mass is treated with a solution of 2-(1-(mercaptomethyl)cyclopropyl)acetic acid of Formula III, also without protecting the carboxyl functional group. Then a solution of sodium methoxide is added as catalyst and subsequently work-up in order to obtain a reaction mixture containing crude Montelukast free acid.

This one-pot straight forward process greatly simplifies the preparation of crude Montelukast free acid. We have found that mixing the reactants in multi-batches mode, preferably drop-wise manner, and conducting the reaction at low temperature, preferably in the range of −15° C. to −10° C., are two of the key factors that make this simple process work.

In this step, the molar ratio of the diol compound of formula II and mesyl chloride and the amine catalyst is crucial in minimizing the formation of process-related impurities, such as the dehygration and cyclic ether impurities. The optimal molar ratio of the amine catalyst and mesyl chloride to diol compound of Formula II ranges within 1.25-1.35 to 1.15-1.25 to 1. This optimized combination leads to the highest yield of Montelukast free acid while keeping the content of process related impurities minimal.

The present invention further provides a method for the isolation of the Montelukast free acid from its reaction mixture. The reaction mixture containing crude Montelukast free acid is quenched with aqueous solution of sodium thiosulfate. Methyl isobutyl ketone (MIBK) is added to form a biphasic system. The organic layer is collected and subsequently washed with aqueous solutions of sodium bicarbonate, tartaric acid, sodium chloride, and sodium thiosulfate. Then this organic layer is dried over anhydrous absorbance and the solvent is distilled off to dryness. The residue is stripped with ethyl acetate. Crude Montelukast free acid is obtained by re-crystallization from a mixed solvent of ethyl acetate and acetonitrile.

Stage II: Preparation of Montelukast-L-ephedrine salt (Formula V) (scheme 2).

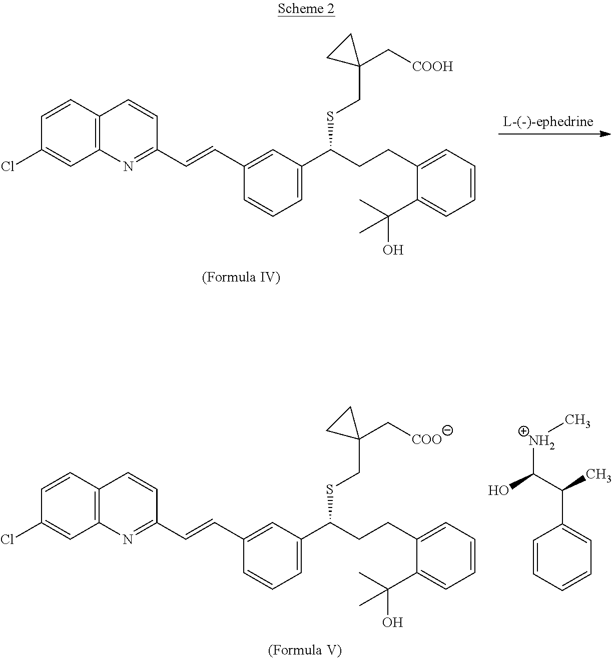

In Stage II, the Montelukast free acid of Formula IV, obtained by the process described in previous stage I, is further purified by means of forming the Montelukast-L-ephedrine salt of Formula V and purifying said salt.

An acetonitrile solution of Montelukast free acid of Formula IV is mixed with a toluene solution of (L)-(−)-ephedrine and heated. Then the reaction mass is cooled down and seeded with Montelukast-(L)-ephedrine salt. The reaction mass is further cooled to facilitate the first crystallization. The crude crystals are re-crystallized using acetonitrile to obtain Montelukast-(L)-ephedrine salt with high chemical and optical purity.

It is already known that the preparation of Montelukast free acid is inevitably accompanied by several by-products due to competitive side reactions. These by-products are difficult to be removed, and therefore, the purification of Montelukast acid requires tedious workup processes, said purification processes increase the production cost and reduce the yield of product. We have found according to the present invention that the L-ephedrine is particular useful in the purification of Montelukast acid and the use of L-ephedrine results in a simple method to obtain Montelukast in high chemical and optical purity and improved yield. In our experiments, Montelukast-L-ephedrine is obtained in yields of about 67.5% and the chemical purity of the product is not less than 99.8% according to the high-performance liquid chromatography method (HPLC) and the un-wanted enantiomer is below detection limit.

Stage III: Preparation of Montelukast Sodium (Formula I) (scheme 3).

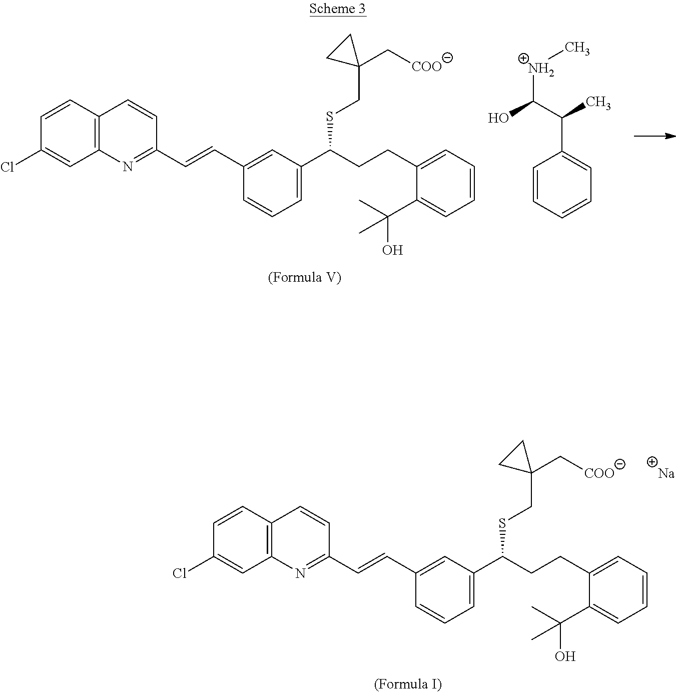

In stage III, the highly pure Montelukast-(L)-ephedrine salt obtained from previous stage II, is converted to highly pure Montelukast acid by treatment with an organic acid, such as acetic acid. Then, said purified Montelukast acid is converted into a pharmaceutical acceptable salt thereof, preferably the sodium salt of Montelukast, by treatment with the corresponding base, preferably the corresponding hydroxide such as sodium hydroxide, in an alcoholic solution. The Montelukast sodium is re-crystallized from cyclohexane/methanol.

It has been found that according to this process, the previously obtained high chemical and optical purity of Montelukast-(L)-ephedrine is also maintained in the product of Montelukast sodium and no additional impurity has been formed in this step and no racemization may take place under the provided conditions.

The process of the present invention will be demonstrated in more details with reference to the following examples, which are provided by way of illustration only and should not be construed as limit to the scope of the reaction in any manner.

### EXAMPLE 1

### Preparation of Montelukast Acid

(S,E)-1-(3-(2-(7-Chloroquinolin-2-yl)vinyl)phenyl)-3-(2-(2-hydroxypropan-2-yl)phenyl)propan-1-ol (210 g, 0.458 mol) and 735 ml tetrahydrofuran (THF) are charged into a 5L 4-neck round bottom flask under inert gas and this mixture is cooled to temperature from about −15° C. to −10° C. Di-isopropyl ethyl amine (74.55 g, 0.577 mol) is added drop-wisely at the same temperature. An additional quantity of 105 ml THF is used to rinse the dropping funnel and the flask and added to the mixture. The stirring is continued for a period of time of about 15 min. Methane sulfonyl chloride (63.06 g, 0.55 mol) is added drop-wisely and the funnel is rinsed with 105 ml THF.

The reaction mixture is further stirred for about 30 min, while maintaining the temperature from about −15° C. to −10° C. Diisopropyl ethyl amine (31.08 g, 0.24 mol) is charged into the above-prepared reaction mass and stirring is maintained for about 4 to 5 hours. 315 ml methyl isobutyl ketone (MIBK) is added slowly to the reaction mixture. To the reaction mixture, a solution of 1-(Mercaptomethyl)cyclopropane acetic acid (167.62 g, 1.147 mol) in 315 ml MIBK is added at temperature from about −15° C. to −10° C. drop-wisely. 120 ml MIBK are used to rinse the funnel. To this reaction mixture, 1260 g Sodium methoxide solution (30% in methanol) are added drop-wisely over a period of 90 minutes, while maintaining the temperature at about −15° C. to −10° C. The reaction mass is stirred further for about 5 hours. Temperature is allowed to rise to about 20° C. and the reaction is further stirred at that temperature for about 5 to 8 hours.

The reaction mass is quenched with 5400 ml 2.5% w/v aqueous solution Sodium thiosulfate at temperature from about 5 to 10° C. To the mass, 3360 ml MIBK is added and layers are separated. To the aqua layer, 1680 ml MIBK is added and layers are stirred and separated. The combined organic layers are washed subsequently with 3150 ml Sodium bicarbonate, 1260 ml 10% w/v aq. tartaric acid solution, 3150 ml Sodium chloride, 3150 ml Sodium thiosulfate 2.5% w/v aq. solution and dried with 210 g Sodium sulfate. The solvent is distilled off under vacuum, while maintaining the temperature below 40° C. and stripped with 265 ml Ethyl acetate. The residue is diluted in 210 ml Ethyl acetate and 788 ml Acetonitrile is added drop-wisely. The obtained solid is filtered on Buchner funnel and wash twice with 158 ml Acetonitrile. The wet cake is unloaded and dried under vacuum at temperature form about 50 to 55° C. for about 10 to 12 hours. Crude Montelucast free acid is obtained with a yield from about 66.4% to 68.55%.

### EXAMPLE 2

### Preparation of Montelukast L-ephedrine Salt

In a 3L 4-neck round-bottom flask, 175 g (0.298 mol) Montelucast acid is diluted in 525 ml Acetonitrile and a solution of 56.73 g (0.343 mol) L-(−)-ephedrine in 200 ml Toluene is added under inert atmosphere. 62.5 ml Toluene is used to rinse the surface of the glassware and added to the mixture. The reaction mixture is heated to temperature from about 45 to 50° C. and 962.5 ml Acetonitrile is charged slowly. The reaction mixture is cooled down to temperature of about 38 to 39° C. and is seeded with 0.88 g (0.001 mol) Montelucast salt. The reaction mixture is further cooled down to temperature from about 30 to 32° C., while maintaining stirring for about 4 to 5 hours. 525 ml Toluene-Acetonitrile mixture (15:85v/v) is added to the mixture and the stirring is continued for about 16 hours. The precipitated solid is filtered on Buchner funnel, washed subsequently with 525 ml and 175 ml Toluene-Acetonitrile mixture (15:85v/v) and sucked dry for about 1 hour. The wet cake is charged back to the flask and 1050 ml Acetonitrile is added.

The reaction mixture is heated to temperature from about 45 to 47° C. for about 1 hour under stirring and is left to cool down to temperature about 30 to 32° C. for a period of about 1 hour. Stirring is maintained for an additional period of about 1 hour. The material is filtered under nitrogen atmosphere, washed subsequently with 175 ml and 135 ml Acetonitrile and sucked dry. The wet cake is unloaded and dry under vacuum at temperature from about 45 to 50° C. for about 15 hours. Purified Montelukast-L-ephedrine is obtained with a yield of 66% to 68.9%.

### EXAMPLE 3

### Preparation of Montelukast Sodium

In a 3L 4-neck r.b. flask, 1500 ml Dichloromethane and 150 g (0.199 mol) Montelukast salt are charged under inert gas. The reaction mass is cooled to temperature from about 15 to 20° C. and extracted twice with about 395 ml Acetic acid 5% w/v aq. solution and washed with 5×667 ml de-mineral water till pH 7. The organic layer is distilled off at temperature below about 32° C. under vacuum and the residue is diluted in 236 ml Methanol and a solution of 8.78 g (0.21 mol) NaOH in 118 ml Methanol is added slowly.

Activated carbon of 2.01 g is charged to the flask, while maintaining the stirring for about 1 hour. The reaction mass is filtered through hyflow bed and washed with 118 ml Methanol. The solvent is distilled off completely at temperature below 45° C. and the residue is stripped with 219 ml Cyclohexane. To the residue 1010 ml Cyclohexane is added and the reaction mixture is stirred for about 1 to 2 hours at temperature from about 25 to 30° C.

The obtained material is filtered on Buchner funnel and washed twice with 100 ml Cyclohexane. The wet cake is charged in 2L Rotavapor flask, followed by 220 ml Methanol. Stirring is maintained for about 15 min and methanol is distilled off completely at temperature below 45° C. under vacuum. The wet material is unloaded and dried under vacuum at temperature from about 65 to 70° C. Montelukast sodium is obtained, i.e. a yield of 86.3% to 86.8%.

The present invention describes a large-scale manufacture process for the preparation of Montelukast sodium with improved yield and purity at relative low production cost in comparison to the prior art processes for producing similar products.

In summary, according to the present invention a one-pot process for the preparation of crude Montelukast free acid is provided, wherein the protection/de-protection steps used in the prior art processes are obviated without sacrificing the yield and quality of the product.

In addition, the use of optically pure L-ephedrine according to the present invention results in a process for the purification of Montelukast free acid, wherein Montelukast-L-ephedrine salt has improved yield with increased chemical and optical purity.

Also the present invention has provided a process for the preparation of Montelukast sodium in improved yield with increased chemical and optical purity from the corresponding Montelukast-L-ephedrine salt.

While the present invention has been described with respect to the particular embodiments, it will be apparent to those skilled in the art that various changes and modifications may be made in the invention without departing from the scope thereof, as defined in the appended claims.

1. A process for the preparation of Montelukast sodium of Formula I, which comprises 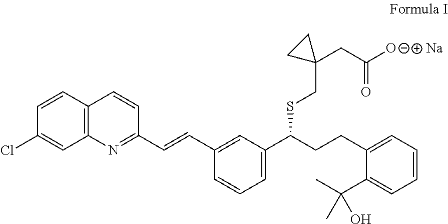
(a) dissolution of {S}-1-[3-[2-(7-chloro-2-quinolinyl)ethyl]phenyl]-3-[2-(1-hydroxyl-1-methylethyl)phenyl]propanol of Formula II in a solvent in the presence of catalyst and addition of mesyl chloride thereto to obtain a reaction mass; 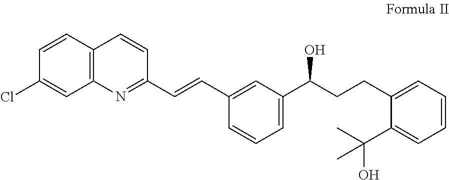
(b) addition of a solution of 2-[1-(mercaptomethyl)cyclopropyl]acetic acid of Formula III into the reaction mass obtained from step (a) followed by a solution of sodium methoxide and work-up to obtain crude Montelukast free acid of Formula IV; 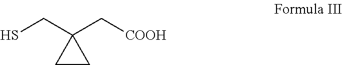
(c) reaction of crude Montelukast free acid of Formula IV with L-ephedrine to obtain crude Montelukast-L-ephedrine of Formula V; 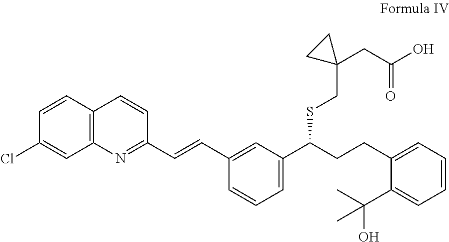
(d) purification of crude Montelukast L-ephedrine salt of Formula V in order to obtain substantial pure Montelukast-L-ephedrine of Formula V with chemical purity of not less than 99.5% and optical purity of not less than 99.9%; 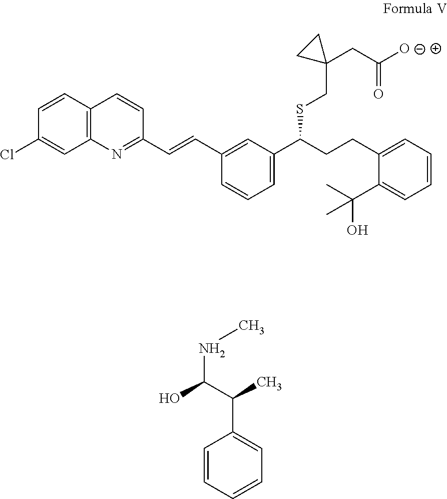
(e) conversion of the substantial pure Montelukast L-ephedrine salt of Formula V into substantial pure Montelukast sodium of Formula I.

2. The process according to claim 1, wherein the catalyst of step (a) is a base, preferably a tertiary amine, most preferably diisopropyl ethyl amine.

3. The process according to claim 1, wherein the solvent of step (a) is a polar aprotic solvent, preferably tetrahydrofuran.

4. The process according to claim 1, wherein the mesyl chloride of step (a) is diluted in an organic solvent, preferably methyl isobutyl ketone, before the addition of step (b).

5. The process according to claim 1, wherein the addition of mesyl chloride of step (a) is conducted in drop-wise manner or multi-batches manner.

6. The process according to claim 1, wherein a base is added prior to the addition of 2-(1-(mercaptomethyl)cyclopropyl)acetic acid of Formula III of step (b), wherein said base is an amine, preferably a tertiary amine, most preferably diisopropyl ethyl amine.

7. The process according to claim 1, wherein the solution of 2-(1-(mercaptomethyl)cyclopropyl)acetic acid of Formula III of step (b) is methyl isobutyl ketone solution.

8. The process according to claim 1, wherein the work-up process of step (b) further comprises the steps of:
(1) quenching the reaction mixture obtained by step (b) with aqueous solution of sodium thiosulfate;
(2) isolating the organic layer and subsequently washed with sodium bicarbonate solution, tartaric acid solution, sodium chloride solution and sodium thiosulfate solution;
(3) concentrating the organic layer to obtain a solid residue;
(4) striping the residue with ethyl acetate; and
(5) re-crystallization using ethyl acetate and acetonitrile.

9. The process according to claim 1, wherein the crude Montelukast acid and L-ephedrine of step (c) is reacted in a mixed solvent of acetonitrile and toluene to obtain Montelukast-L-ephedrine salt.

10. The process according to claim 1, wherein the purification of step (d) is re-crystallization using a single solvent, preferably a polar aprotic solvent, such as acetonitrile.

11. The process according to claim 1, wherein the conversion of step (e) further comprises the steps of:
(i) treating the Montelukast-L-ephedrine salt obtained from step (d) in claim 1 with an organic acid, such as acetic acid;
(ii) basifying the Montelukast acid with sodium hydroxide in an alcoholic solution; and
(iii) re-crystallization to obtain Montelukast sodium of Formula I in high chemical and optical purity.

12. The process according to claim 11, wherein the substantially pure Montelukast acid of step (i) is obtained by preparing a dichloromethane solution of the substantially pure Montelukast-L-ephedrine and wash said solution with acetic acid aqueous solution followed by distilling off the organic solvent.

13. The process according to claim 11, wherein the basifying reaction of step (ii) is carried out using sodium hydroxide in methanol solution.

14. The process according to claim 11, wherein the re-crystallization of step (iii) is conducted in cyclohexane.
